(19) United States
(12) Patent Application Publication
(10) Pub. No.: US 2021/0219817 A1
(43) Pub. Date: Jul. 22, 2021

(54) MEASUREMENT APPARATUS, MEASUREMENT METHOD, AND RECORDING MEDIUM

(71) Applicant: OLYMPUS CORPORATION, Tokyo (JP)

(72) Inventors: Yugo KOIZUMI, Tokyo (JP); Misa HIROSE, Tokyo (JP)

(73) Assignee: OLYMPUS CORPORATION, Tokyo (JP)

(21) Appl. No.: 17/220,292

(22) Filed: Apr. 1, 2021

Related U.S. Application Data
(63) Continuation of application No. PCT/JP2018/037003, filed on Oct. 3, 2018.

(51) Int. Cl. A61B 1/00; A61B 1/31; A61B 1/015

(52) U.S. Cl. CPC A61B 1/00006; A61B 1/00009; A61B 1/015; A61B 1/31; A61B 1/00039

(57) ABSTRACT

A measurement apparatus includes a time measurement unit that measures an observation time from when a distal end portion of an endoscope arrives at the predetermined site in a subject to when an endoscopy ends.

### CROSS-REFERENCE TO RELATED APPLICATIONS

This application is a continuation application of International Application PCT/JP2018/037003 filed on Oct. 3, 2018, the entire contents of which are incorporated herein by reference.

### TECHNICAL FIELD

The present disclosure relates to a measurement apparatus, a measurement method, and a recording medium.

### BACKGROUND

As a system used in endoscopy, for example, an electronic endoscope system capable of automatically and accurately acquiring a use situation of an electronic endoscope has been proposed (refer to Japanese Patent Laid-Open No. 2002-345726). This electronic endoscope system includes endoscope mounting determination means for determining whether or not an electronic endoscope is mounted on a processor for endoscope, and a recording device that records the use situation of the electronic endoscope, the recording device including endoscope mounting time recording means for measuring a time period during which the electronic endoscope is mounted on the processor for endoscope based on a determination result by the endoscope mounting determination means and recording a cumulative time period during which the electronic endoscope is mounted on the processor for endoscope.

### SUMMARY

According to an aspect of the present disclosure, there is provided a measurement apparatus including a processor connected to a memory. The processor is programmed to function as a first detection unit configured to detect a first time, the first time being a time that a distal end portion of an endoscope arrives at a predetermined site in a subject during an endoscopy. The processor is further programmed to function as a second detection unit configured to detect a second time, the second time being a time that the endoscopy ends. The processor is further programmed to function as a time measurement unit that measures a first time period beginning at the first time and ending at the second time.

According to another aspect of the present disclosure, there is provided a measurement method including measuring, after endoscopy is started, a first time period as a time period elapsed from when a distal end portion of an endoscope arrives at a predetermined site in a subject until the endoscopy ends, measuring a second time period, the second time period being a time period within the first time period during which an inside of the subject is subjected to a treatment, and calculating a difference between the first time period and the second time period as an observation time period.

According to yet another aspect of the present disclosure, there is provided a non-transitory, computer-readable recording medium having a program recorded thereon, the program causing a measurement unit that measures an observation time period of endoscopy to perform processing including a step of measuring a first time period as a time period elapsed from when a distal end portion of an endoscope arrives at a predetermined site in a subject until the endoscopy ends, a step of measuring a second time period, the second time period being a time period within the first time period during which an inside of the subject is subjected to a treatment, and a step of calculating a difference between the first time period and the second time period as the observation time period.

### BRIEF DESCRIPTION OF DRAWINGS

FIG. 1 is a diagram illustrating a configuration example of an endoscope system according to a first embodiment.

FIG. 2 is a diagram for describing an example of an observation time period to be measured by a processor during colonoscopy.

FIG. 3 is a diagram illustrating an example of an endoscope system according to a second embodiment.

FIG. 4 is a diagram illustrating an endoscope system according to a modification of the second embodiment.

FIG. 5 is a diagram illustrating an example of a hardware configuration.

### DETAILED DESCRIPTION

In recent endoscopy, the quality of examination is required to be demonstrated.

For example, in colonoscopy, after a distal end portion of an endoscope inserted into a subject through the anus has arrived at the cecum, observation is performed while the endoscope is being extracted. Thus, a demonstration that the distal end portion of the endoscope has arrived at the cecum and a demonstration that the observation has been performed by extracting the endoscope over a sufficiently long time period are required.

However, it has taken time and effort to record data for performing the demonstrations.

In view of such actual circumstances, there are provided a measurement apparatus, a measurement method, and a recording medium, which enable a time period required for observation in endoscopy to be measured, in embodiments disclosed below.

The embodiments will be described below with reference to the drawings.

### First Embodiment

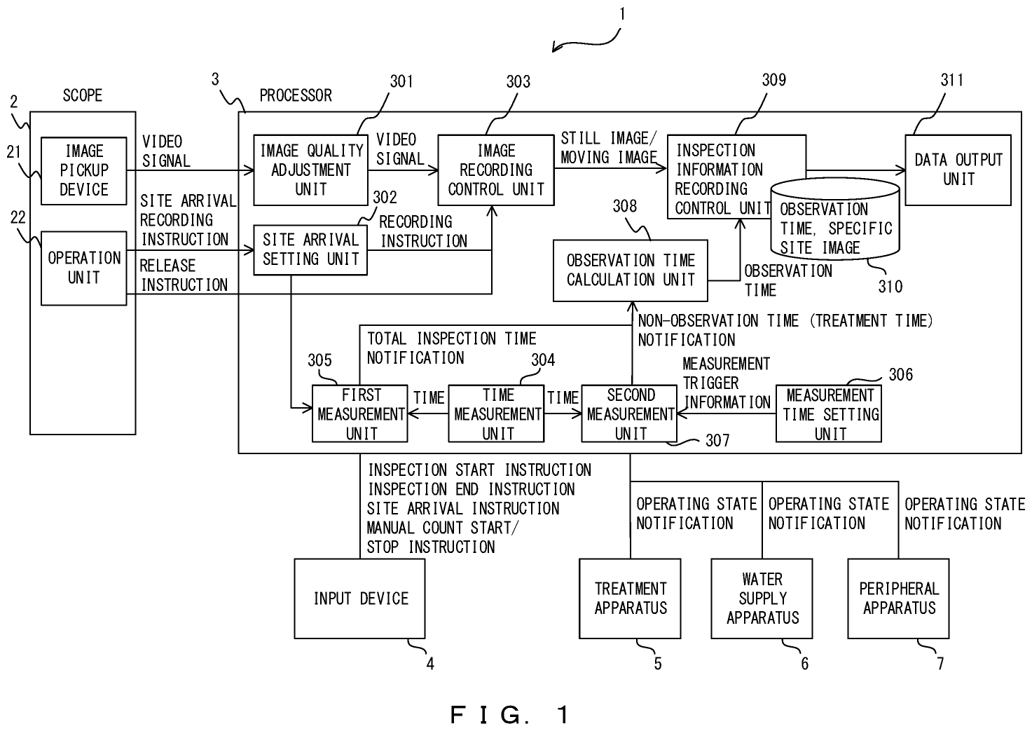

FIG. 1 is a diagram illustrating a configuration example of an endoscope system according to a first embodiment.

As illustrated in FIG. 1, an endoscope system 1 includes a scope 2, a video processor for endoscope (hereinafter referred to as a “processor”) 3, an input device 4, a treatment apparatus 5, a water supply apparatus 6, and a peripheral apparatus 7. The processor 3 is an example of an endoscope apparatus here. The treatment apparatus 5 and the water supply apparatus 6 are each an example of a peripheral apparatus. The peripheral apparatus 7 is a peripheral apparatus other than the treatment apparatus 5 and the water supply apparatus 6.

Each of the scope 2, the input device 4, the treatment apparatus 5, the water supply apparatus 6, and the peripheral apparatus 7 is connected to the processor 3. For example, each of the treatment apparatus 5, the water supply apparatus 6, and the peripheral apparatus 7 is connected to the processor 3 via a cable.

The scope 2 is a flexible endoscope.

The scope 2 includes an image pickup device 21 in its distal end portion, and picks up an image of the inside of a subject using the image pickup device 21 and outputs a video signal corresponding to a result of the image pickup to the processor 3. The image pickup device 21 is an image sensor such as a CCD (charge coupled device) or a CMOS (complementary metal oxide semiconductor).

The scope 2 includes an operation unit 22. The operation unit 22 includes a plurality of operation buttons (also referred to as “operation switches”) including a site arrival recording button and a release button. The site arrival recording button is a button for issuing an instruction to record an observation image when a distal end portion of the scope 2 (hereinafter referred to as a “scope distal end portion”) has arrived at a specific site in the subject. The release button is a button for issuing a release instruction. In the operation unit 22, a site arrival recording instruction signal is outputted to the processor 3 when the site arrival recording button is pressed, and a release instruction signal is outputted to the processor 3 when the release button is pressed.

The input device 4 is a keyboard or a medical tablet connected to be communicable with the processor 3, for example, and performs various types of inputs to the processor 3 in response to a user's operation. For example, instruction signals respectively representing an examination start instruction, an examination end instruction, a site arrival instruction, a manual count start/stop instruction, and the like are inputted.

The treatment apparatus 5 is an apparatus that performs a treatment for a target site in the subject, for example, an electrocautery apparatus. The electrocautery apparatus dissects or coagulates the target site, for example, by outputting a high-frequency current to an electrocautery inserted into the scope 2.

The water supply apparatus 6 supplies liquid such as water to the scope distal end portion via a water supply tube in the scope 2 and releases the liquid such as water to the outside for the purpose of ensuring an observation visual field and cleaning an observation site, for example.

The peripheral apparatus 7 is an air supply apparatus, a printer, or the like. The air supply apparatus supplies air such as carbon dioxide to the scope distal end portion via an air supply tube in the scope 2 and releases the air to outside for the purpose of ensuring the observation visual field and cleaning the observation site, for example, like the water supply apparatus 6. The printer prints examination information or the like.

Each of the treatment apparatus 5, the water supply apparatus 6, and the peripheral apparatus 7 notifies its operating state indicating whether or not itself is operating to the processor 3.

The processor 3 includes an image quality adjustment unit 301, a site arrival setting unit 302, an image recording control unit 303, a time measurement unit 304, a first measurement unit 305, a measurement time setting unit 306, a second measurement unit 307, an observation time calculation unit 308, an examination information recording control unit 309, an examination information recording unit 310, and a data output unit 311. Each of the time measurement unit 304, the first measurement unit 305, the second measurement unit 307, and the observation time calculation unit 308 is an example of a time measurement unit that measures an observation time period during endoscopy.

The image quality adjustment unit 301 performs image quality adjustment processing for a video signal inputted from the scope 2, and outputs the video signal that has been subjected to the image quality adjustment processing to the image recording control unit 303. The video signal that has been subjected to the image quality adjustment processing is also outputted to a monitor for endoscope not illustrated connected to the processor 3, and displays an image (observation image) corresponding to the video signal on the monitor for endoscope.

The site arrival setting unit 302 previously holds information representing a correspondence between a determination how many times the site arrival recording instruction signal has been inputted from the scope 2 since the examination was started and an arrival site (hereinafter referred to as “correspondence information”) in a memory not illustrated. When the site arrival recording instruction signal is inputted from the scope 2, a recording instruction signal of a corresponding arrival site is outputted to the image recording control unit 303 depending on how many times the site arrival recording instruction signal has been inputted since the examination was started, and the corresponding arrival site is notified to the first measurement unit 305.

Assuming colonoscopy, for example, the site arrival setting unit 302 previously holds as correspondence information correspondence information indicating that the cecum is associated with the first site arrival recording instruction signal, the right colic flexure is associated with the second site arrival recording instruction signal, and the left colic flexure is associated with the third site arrival recording instruction signal, . . . . In this case, after the colonoscopy is started, the recording instruction signal associated with the cecum, the recording instruction signal associated with the right colic flexure, the recording instruction signal associated with the left colic flexure, . . . are outputted to the image recording control unit 303 in order from the site arrival setting unit 302 by a user pressing the site arrival recording button in the operation unit 22 every time the scope distal end portion arrives at a specific site such as the cecum, the right colic flexure, the left colic flexure, . . . , and the cecum, the right colic flexure, the left colic flexure, . . . are notified as an arrival site to the first measurement unit 305.

The user can freely change a content of the correspondence information to be held in the site arrival setting unit 302 depending on respective operations of the input device 4 and front panels not illustrated in the processor 3, for example. The site arrival setting unit 302 may hold the correspondence information for each type of examination, or may select the correspondence information to be used in response to examination to be performed by the user.

The image recording control unit 303 generates an image for recording (a still image or a moving image) based on the video signal inputted from the image quality adjustment unit 301 and outputs the generated image to the examination information recording control unit 309 when the recording instruction signal of the arrival site is inputted from the site arrival setting unit 302 or the release instruction signal is inputted from the scope 2. An image generated when the recording instruction signal of the arrival site is inputted is accompanied by arrival site information indicating what site is the arrival site. The image accompanied by the arrival site information is also referred to as a specific site image.

The time measurement unit 304 generates time information representing a current time, and outputs the generated time information to the first measurement unit 305 and the second measurement unit 307.

The first measurement unit 305 measures a total examination time period as a time period from an examination start time to an examination end time based on the instruction signal to be inputted from the input device 4, the arrival site to be notified from the site arrival setting unit 302, and the time information to be inputted from the time measurement unit 304, and notifies the total examination time period to the observation time calculation unit 308. The examination start time to be used to measure the total examination time period is a time considered as an observation start time, and is a time represented by the time information inputted from the time measurement unit 304 when an examination start instruction signal from the input device 4, a site arrival instruction signal representing an observation start site from the input device 4, a manual count start instruction signal from the input device 4, or an arrival site representing an observation start site from the site arrival setting unit 302 is inputted or notified. The observation start site is the cecum in colonoscopy, for example. The examination end time to be used to measure the total examination time period is a time represented by the time information inputted from the time measurement unit 304 when an examination end instruction signal from the input device 4, a site arrival instruction signal representing an examination end site from the input device 4, a manual count stop instruction signal from the input device 4, or an arrival site representing an examination end site from the site arrival setting unit 302 is inputted or notified.

The first measurement unit 305 may be configured such that the measurement of the total examination time can be stopped or resumed on the way using a manual count start/stop instruction signal to be inputted from the input device 4. In this case, the first measurement unit 305 may stop the measurement of the total examination time period when a manual count stop instruction from the input device 4 is inputted during the measurement and then resume the measurement when the manual count start instruction from the input device 4 is inputted.

The measurement time setting unit 306 previously holds measurement trigger information representing a measurement trigger of a non-observation time period to be measured by the second measurement unit 307 in a memory not illustrated, and outputs the measurement trigger information to the second measurement unit 307. In the present embodiment, the measurement trigger information indicates that the time when any one of the treatment apparatus 5, the water supply apparatus 6, and the peripheral apparatus 7 in a non-operating state has shifted to an operating state is set as a measurement start trigger and the time when all the treatment apparatus 5, the water supply apparatus 6, and the peripheral apparatus 7 have shifted to a non-operating state is set as a measurement end trigger.

A content of the measurement trigger information to be held in the measurement time setting unit 306 can be freely changed by the user depending on the respective operations of the input device 4 and the front panels in the processor 3, for example.

The second measurement unit 307 measures the non-observation time period in the total examination time period to be measured by the first measurement unit 305 based on the measurement trigger information inputted from the measurement time setting unit 306, the respective operating states to be notified from the treatment apparatus 5, the water supply apparatus 6, and the peripheral apparatus 7, and the time information to be inputted from the time measurement unit 304, and notifies the non-observation time period to the observation time calculation unit 308. The non-observation time period means a time period not required for observation. In the present embodiment, a treatment time period for the inside of the subject, such as a time period during which a treatment is being performed by the treatment apparatus 5, a time period during which water supply is being performed by the water supply apparatus 6, or a time period during which air supply is being performed by the air supply apparatus as the peripheral apparatus 7 is set as a non-observation time period. That is, a time period during which any one of the treatment apparatus 5, the water supply apparatus 6, and the peripheral apparatus 7 is operating is set as a non-observation time period.

The observation time calculation unit 308 calculates a difference between the total examination time period notified from the first measurement unit 305 and the non-observation time period notified from the second measurement unit 307, and notifies the difference as an observation time period to the examination information recording control unit 309. The calculated difference represents a time period obtained by subtracting the non-observation time period from the total examination time period, i.e., a time period required for observation in the total examination time period.

The examination information recording control unit 309 records on the examination information recording unit 310 the image or the specific site image inputted from the image recording control unit 303 and the observation time period notified from the observation time calculation unit 308 in association with each other. The examination information recording control unit 309 outputs the image or the specific site image recorded in association with the examination information recording unit 310 and the observation time period to the data output unit 311.

The examination information recording unit 310 is a nonvolatile recording device, e.g., a HDD (hard disk drive).

The data output unit 311 outputs the image or the specific site image and the observation time period, which are associated with each other, inputted from the examination information recording control unit 309, to an external memory not illustrated removably mounted on the processor 3, a server not illustrated connected to the processor 3 via a hospital LAN (local area network) not illustrated, or the like. The external memory is a USB (universal serial bus) memory or the like.

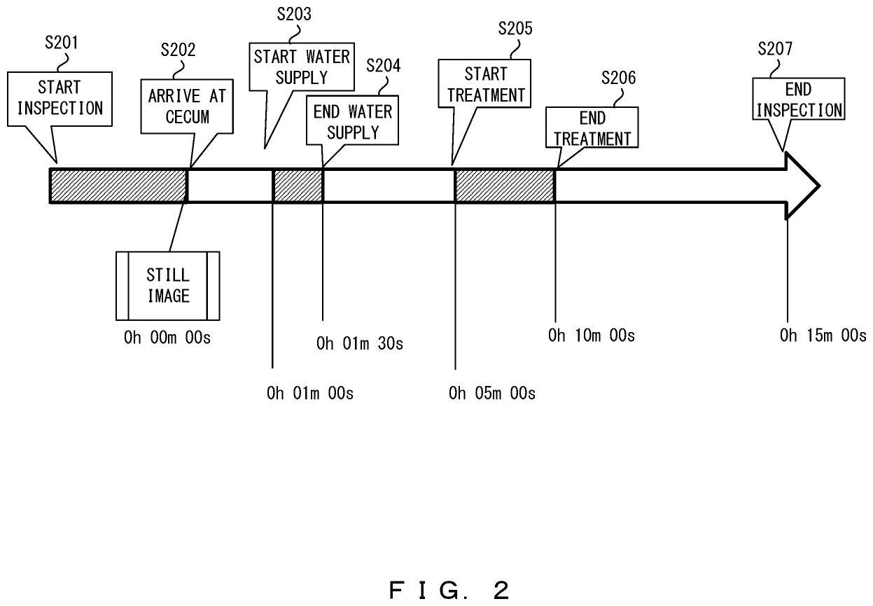

FIG. 2 is a diagram for describing an example of an observation time period measured in the processor 3 during colonoscopy.

In the example illustrated in FIG. 2, the colonoscopy is started (S201), and the scope 2 is inserted into the subject through the anus. When the scope distal end portion arrives at the cecum, observation is started while the scope 2 is being extracted.

First, if the user presses, when the scope distal end portion has arrived at the cecum (S202), the site arrival recording button in the operation unit 22 in the scope 2 or operates the input device 4 to issue an examination start instruction, a cecum arrival instruction, or a manual count start instruction, a specific site image (still image) of the cecum is generated by the image recording control unit 303, and the measurement of the total examination time period by the first measurement unit 305 is started.

Then, it is assumed that water supply by the water supply apparatus 6 and the treatment by the treatment apparatus 5 have been performed. At this time, a time period required for the water supply by the water supply apparatus 6 and the treatment by the treatment apparatus 5 is measured as a non-observation time period by the second measurement unit 307. A time period required for the water supply by the water supply apparatus 6 is a time period from the start of the water supply (S203) to the end of the water supply (S204), i.e., a time period during which the water supply apparatus 6 is in an operating state. A time period required for the treatment by the treatment apparatus 5 is a time period from the start of the treatment (S205) to the end of the treatment (S206), i.e., a time period during which the treatment apparatus 5 is in an operating state.

When the user issues an examination end instruction, for example, depending on the operation of the input device 4 (S207), for example, the colonoscopy ends.

In the example illustrated in FIG. 2, assuming that the time when the scope distal end portion arrives at the cecum and the site arrival recording button in the operation unit 22 has been pressed is “0 h00 m00 s”, for example, and the time when the examination end instruction has been issued depending on the operation of the input device 4 is “0 h15 m00 s”, for example, the total examination time measured by the first measurement unit 305 is 15 minutes.

On the other hand, assuming that a water supply start time is “0 h01 m00 s”, a water supply end time is “0 h01 m30 s”, a treatment start time is “0 h05 m00 s”, and a treatment end time is “0 h10 m00 s”, the non-observation time period measured by the second measurement unit 307 is five minutes and thirty seconds that is the sum of 30 seconds as a water supply time period and 5 minutes as a treatment time period.

In this case, the observation time period calculated by the observation time calculation unit 308 is nine minutes and thirty seconds as a difference between 15 minutes as a total examination time period and five minutes and thirty seconds as a non-observation time period. The observation time period and the specific site image (still image) of the cecum generated in S202 are associated with each other and are recorded on the examination information recording unit 310.

As described above, according to the first embodiment, the specific site image is recorded on the examination information recording unit 310 so that the arrival at the specific site, together with the image, can be recorded. Thus, it can be demonstrated that examination target sites have been observed without any omission.

An observation time period elapsed since the scope distal end portion has arrived at a specific site as an observation start site can also be recorded on the examination information recording unit 310. Thus, it can also be demonstrated that it takes sufficiently long time for observation during examination.

In the present embodiment, the following modification can be made.

For example, the measurement of the non-observation time period by the second measurement unit 307 may be performed based on an operation signal in each of operation units provided in the endoscope system 1. Examples of the operation unit include the operation unit 22 in the scope 2, the front panel in the processor 3, the input device 4 that is a keyboard, the touch panel provided in the input device 4 that is a medical tablet, and a foot switch not illustrated connected to each of any one or more of the processor 3, the treatment apparatus 5, the water supply apparatus 6, and the peripheral apparatus 7.

For example, the release button may be made to have a function of the site arrival recording button in the operation unit 22 in the scope 2. In this case, the user presses the release button every time the scope distal end portion arrives at the specific site so that an instruction to record the specific site is outputted to the image recording control unit 303 and the specific site is notified to the first measurement unit 305.

For example, an examination end time used to measure the total examination time period by the first measurement unit 305 may be a time represented by time information inputted from the time measurement unit 304 when the scope 2 is removed from inside the subject. In this case, detection of the removal may be performed based on the luminance of an image represented by the video signal to be outputted by the scope 2. For example, when a value of the luminance reaches a predetermined value or more, it may be detected that the scope 2 has been removed from inside the subject. In this case, the observation time period calculated by the observation time calculation unit 308 can also be considered as a time period related to a removal operation of the scope 2 in the total examination time period measured by the first measurement unit 305.

### Second Embodiment

Conventionally, as an example of a function of an endoscope system, an endoscope system having a function of recording a still image and a time at the time of arrival of an examination event such as scope insertion and polyp removal to be performed during examination has been known.

The function is implemented when IT (information technology) equipment such as a PC (personal computer) records a time and a still image obtained by capturing an endoscope observation screen represented by a video signal that has been subjected to streaming transmission from a processor via a video signal cable in association with each other with an examination event recording instruction signal received from the processor as a trigger.

The function enables a user to confirm a series of flows during examination in a time series and to also use its recording content to evaluate a quality of the examination, for example. In the function, when the user registers the presence or absence of still image recording for each examination event in the IT equipment in advance, a recording method for each examination event can also be selected.

On the other hand, to perform large-capacity communication between the processor and the IT equipment, there is a demand to unify communication between the processor and the IT equipment into Ethernet communication.

However, when the demand has been met, various problems may occur.

When the above-described streaming transmission is implemented by Ethernet communication, for example, communication in a hospital LAN is compressed so that a still image may be difficult to record by a capture in the IT equipment. As a result, when the still image cannot be recorded in the IT equipment, the still image may be difficult to record in a recording method selected for each examination event. Further, a time lag occurs between a timing at which the user has issued an examination event recording instruction and a timing at which the IT equipment has received its instruction signal, and a correct time may be difficult to record in the IT equipment.

The second embodiment proposes an endoscope system capable of unifying communication between the processor and the IT equipment into Ethernet communication while solving the problems.

In the description of the second embodiment, the same elements as the elements described in the first embodiment are respectively assigned the same reference numerals.

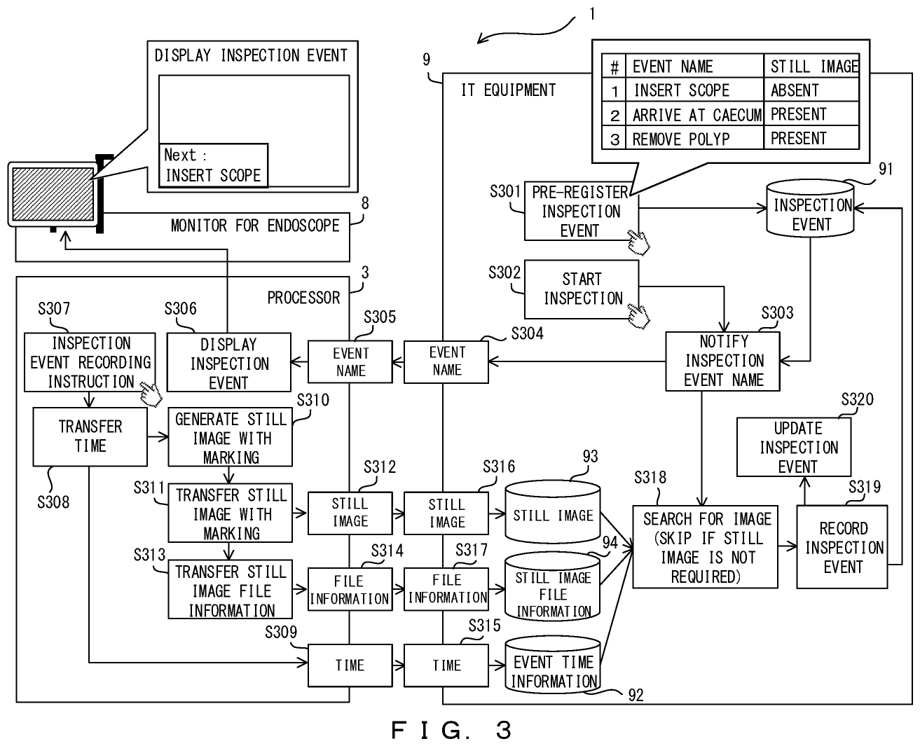

FIG. 3 is a diagram illustrating an example of an endoscope system according to the second embodiment.

The endoscope system 1 illustrated in FIG. 3 includes a processor 3, a monitor for endoscope 8, and IT equipment 9. The processor 3 and the monitor for endoscope 8 are connected to each other via a cable, for example. The processor 3 and the IT equipment 9 are connected to each other via a hospital LAN. That is, communication between the processor 3 and the IT equipment 9 is unified into Ethernet communication.

The monitor for endoscope 8 is an LCD (liquid crystal display), for example.

The IT equipment 9 is a PC, for example.

In the endoscope system 1 illustrated in FIG. 3, the presence or absence of still image recording for each examination event to be performed during endoscopy is registered by a user in advance in the IT equipment 9 (S301). The registration is performed depending on an operation of an input device such as a keyboard included in the IT equipment 9, for example. A registered content is recorded as registration information on an examination event recording unit 91 included in the IT equipment 9. The examination event recording unit 91 is a nonvolatile recording device, e.g., an HDD.

Then, in the IT equipment 9, when the user issues an endoscopy start instruction (S302), a name of an examination event to be first implemented during examination is notified to the processor 3 based on registration information recorded on the examination event recording unit 91 (S303, S304). The endoscopy start instruction is issued depending on an operation of the input device included in the IT equipment 9, for example.

When the processor 3 is notified of the examination event name from the IT equipment 9 (S305), the processor 3 displays the examination event name on the monitor for endoscope 8 as a name of an examination event to be next implemented (S306).

In the processor 3, when the user issues an examination event recording instruction at a time point where the examination event of the name displayed on the monitor for endoscope 8 has been implemented (S307), a time at the time point is transferred to the IT equipment 9 (S308, S309). A still image with marking obtained by marking a still image as an endoscope observation image at the time point is generated (S310). The examination event recording instruction is issued depending on an operation of a front panel in the processor 3 or an input device such as a keyboard, for example. The marking added to the still image is information indicating that the still image is a still image at the time of arrival of the examination event.

When the generation of the still image with marking (S310) ends, the still image with marking is transferred to the IT equipment 9 (S311, S312), and file information related to the still image with marking is transferred to the IT equipment 9 (S313, S314). Examples of the file information related to the still image with marking include information indicating which type of examination event has arrived when the still image has been generated and information about the time transferred in S308.

In the IT equipment 9, when the time is transferred from the processor 3 (S315), the time is recorded on an event time recording unit 92 included in the IT equipment 9. When the still image with marking is transferred from the processor 3 (S316), the still image with marking is recorded on a still image recording unit 93 included in the IT equipment 9. When file information is transferred from the processor 3 (S317), the file information is recorded on a still image file information recording unit 94 included in the IT equipment 9. All the event time recording unit 92, the still image recording unit 93, and the still image file information recording unit 94 are each a nonvolatile recording device, e.g., an HDD.

In the IT equipment 9, it is determined whether or not still image recording is required when the examination event of the name notified in S303 has arrived based on the registration information recorded on the examination event recording unit 91. If a result of the determination is that the still image recording is required, the examination event, the time, and the still image with marking recorded on the still image recording unit 93 are associated with one another and are recorded as examination event information on the examination event recording unit 91 based on the time recorded on the event time recording unit 92 and the file information recorded on the still image file information recording unit 94 (S318, S319). On the other hand, if the determination result is that the still image recording is not required, only the examination event and the time are associated with one another and are recorded as examination event information on the examination event recording unit 91 (S319).

When the recording of the examination event information (S319) ends, the examination event is updated to a subsequent examination event in the IT equipment 9 (S320).

Thereafter, processing in S303 and subsequent steps is performed in a similar manner for the updated examination event.

As described above, according to the second embodiment, only when the user has issued the examination event recording instruction, data is transferred to the IT equipment 9 from the processor 3, and the data is only the time, the still image with marking, and the file information. Therefore, even if communication between the processor 3 and the IT equipment 9 is unified into Ethernet communication, the still image at the time of arrival of the examination event can be recorded without communication in the hospital LAN being compressed. It goes without saying that the still image can be recorded depending on the presence or absence of the still image recording for each examination event registered in the registration information.

When the user has issued the examination event recording instruction, the time acquired in the processor 3 is transferred to the IT equipment 9. Thus, a correct examination event arrival time can be recorded without being affected by a time lag due to Ethernet communication.

In the second embodiment, the following modification can be made.

Although in the endoscope system 1 illustrated in FIG. 3, it is determined whether or not the still image recording is required in the IT equipment 9, for example, the endoscope system 1 may be modified to a configuration in which the determination is performed in the processor 3.

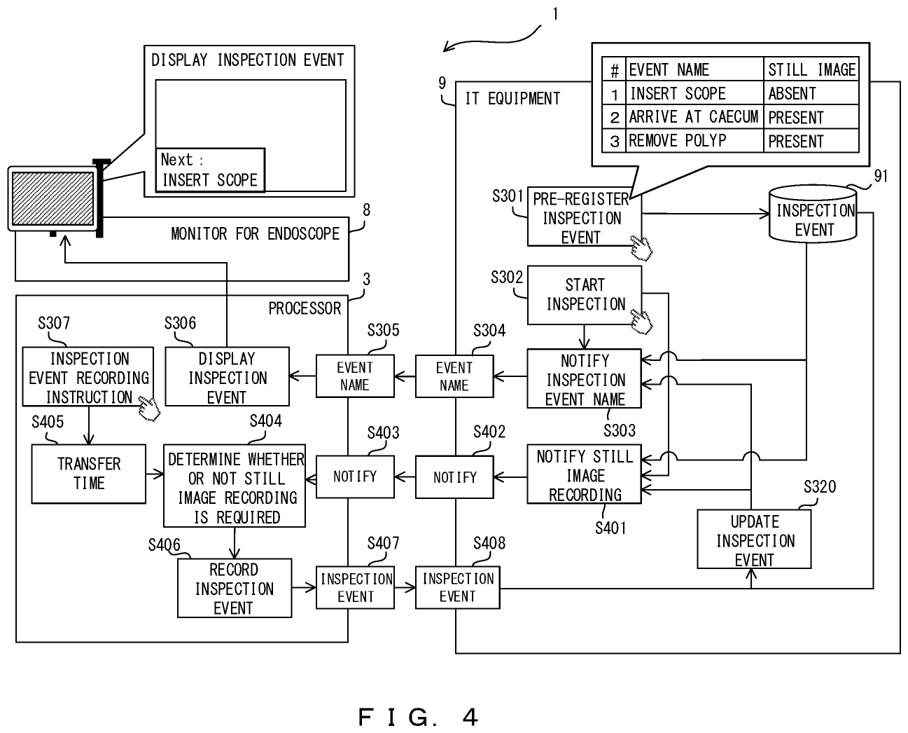

FIG. 4 is a diagram illustrating an endoscope system according to the modification.

In the endoscope system 1 illustrated in FIG. 4, pre-registration is performed by a user, like in the endoscope system 1 illustrated in FIG. 3, in IT equipment 9 (S301).

Then, in the IT equipment 9, when a user issues an endoscopy start instruction (S302), an examination event name is notified to a processor 3 (S303, S304), like in the endoscope system 1 illustrated in FIG. 3. In addition, the presence or absence of still image recording at the time of arrival of the examination event of the name notified in S303 is notified to the processor 3 based on registration information recorded on an examination event recording unit 91 (S401, S402).

When the processor 3 is notified of the examination event name from the IT equipment 9 (S305), the examination event name is displayed on a monitor for endoscope 8 (S306), like in the endoscope system 1 illustrated in FIG. 3. When the presence or absence of the still image recording is notified from the IT equipment 9 (S403), it is determined whether or not the still image recording is required based on the notification (S404).

In the processor 3, if the user performs the examination event of the name displayed on the monitor for endoscope 8 to issue an examination event recording instruction (S307) when a result of the determination in S404 is that the still image recording is required, a time at the time point, a still image with marking obtained by marking a still image as an endoscope observation image at the time point, and the examination event are associated with one another, are transmitted as examination event information to the IT equipment 9, and are recorded on the examination event recording unit 91 in the IT equipment 9 (S404, 5405, S406, S407, S408).

On the other hand, if the user issues the examination event recording instruction when the determination result in S404 is that the still image recording is not required (S307), only a time at the time point and the examination event are associated with each other, are transmitted as examination event information to the IT equipment 9, and are recorded on the examination event recording unit 91 in the IT equipment 9 (S404, 5405, S406, S407, S408).

In the IT equipment 9, when the recording of the examination event information on the examination event recording unit 91 ends, the examination event is updated to a subsequent examination event (S320), like in the endoscope system 1 illustrated in FIG. 3.

Thereafter, processing in S303 and subsequent steps and processing in S401 and subsequent steps are performed in a similar manner for the updated examination event.

As described above, according to the modification, the determination whether or not the still image recording is required, which has been performed in the IT equipment 9, can be performed in the processor 3.

Although the first and second embodiments have been described above, a configuration of a part of the endoscope system 1 according to each of the embodiments may be implemented by a hardware configuration described below.

For example, each of the units in the processor 3 illustrated in FIG. 1, a configuration in which the processing to be performed by the processor 3 is performed, which has been described with reference to FIG. 3, and a configuration in which the processing to be performed by the processor 3 is performed, which has been described with reference to FIG. 4, may be implemented by a circuit such as an FPGA (field-programmable gate array) or an ASIC (application specific integrated circuit).

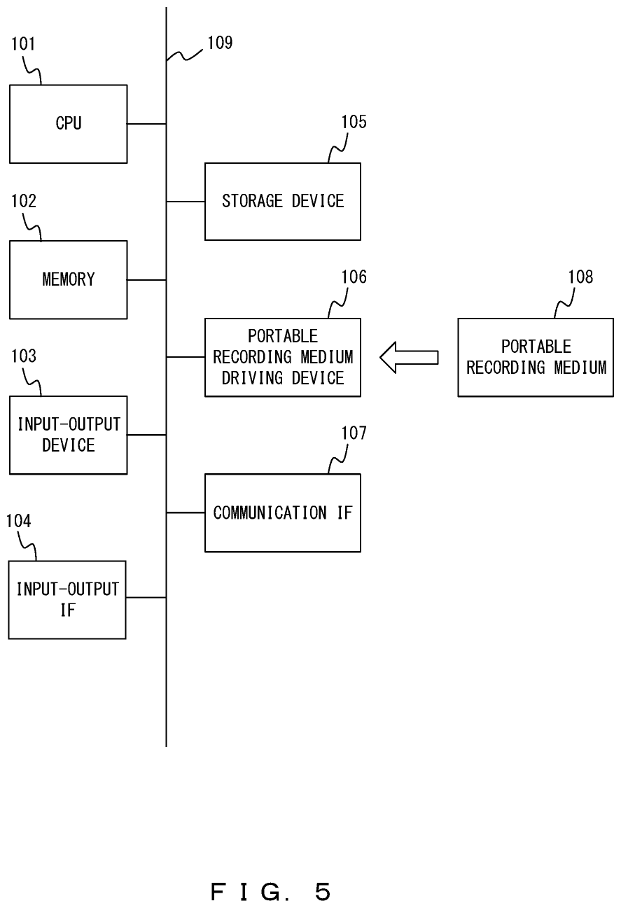

For example, each of the units in the processor 3 illustrated in FIG. 1, a configuration in which the processing to be performed by the processor 3 is performed and a configuration of the IT equipment 9, which have been described with reference to FIG. 3, and a configuration in which the processing to be performed by the processor 3 is performed and a configuration of the IT equipment 9, which have been described with reference to FIG. 4, may be implemented by a hardware configuration illustrated in FIG. 5.

FIG. 5 is a diagram illustrating an example of a hardware configuration.

The hardware configuration illustrated in FIG. 5 includes a CPU (central processing unit) 101, a memory 102, an input-output device 103, an input-output IF (interface) 104, a storage device 105, a portable recording medium driving device 106 that houses a portable recording medium 108, and a communication IF 107, which are connected to one another via a bus 109.

The CPU 101 is an operation device that executes a program for processing to be performed by the processor 3 or the IT equipment 9. The memory 102 is a RAM or a ROM. The RAM is used as a work area of the CPU 101, for example, and the ROM stores a program and information required to execute the program in a nonvolatile manner.

The input-output device 103 includes an input device such as a touch panel or a keyboard and an output device such as a display device.

The input-output IF 104 is an interface for transmitting and receiving a signal between an external apparatus and itself. If an apparatus having the hardware configuration illustrated in FIG. 5 is the processor 3, for example, examples of the external apparatus include the scope 2, the input device 4, the treatment apparatus 5, the water supply apparatus 6, the peripheral apparatus 7, and the monitor for endoscope 8.

The storage device 105 is a storage storing a program and information required to execute the program, information acquired by the execution of the program, and the like in a nonvolatile manner. An example of the storage device 105 is a HDD. The portable recording medium driving device 106 drives the portable recording medium 108 and accesses its recording content. Examples of the portable recording medium 108 is a memory device, a flexible disk, an optical disk, and a magneto-optical disk. Examples of the portable recording medium 108 include a CD-ROM (compact disk read only memory), a DVD (digital versatile disk), and a USB memory. The portable recording medium 108 is also a storage storing a program and information required to execute the program, information acquired by the execution of the program, and the like in a nonvolatile manner, like the storage device 105.

The communication IF 107 is an interface to be connected to a hospital LAN and for communicating with the external apparatus via the hospital LAN.

The present application is not limited to the above-described embodiments as they are, but may be embodied by modifying constituent elements without departing from the scope and spirit of the application in an implementation stage. Various inventions can be formed by an appropriate combination of a plurality of constituent elements disclosed in the above-described embodiments. For example, some of all the constituent elements described in the embodiments may be deleted. Further, the constituent elements in the different embodiments may be appropriately combined.

What is claimed is:

1. A measurement apparatus comprising:
a processor connected to a memory, the processor programmed to function as:
a first detection unit configured to detect a first time, the first time being a time that a distal end portion of an endoscope arrives at a predetermined site in a subject during an endoscopy;
a second detection unit configured to detect a second time, the second time being a time that the endoscopy ends; and
a time measurement unit that measures a first time period beginning at the first time and ending at the second time.

2. The measurement apparatus according to claim 1, wherein
the second time is a time at which the second detection unit determines that the endoscope has been removed from the subject.

3. The measurement apparatus according to claim 1, wherein
the first time is a time at which the first detection unit receives an observation image of the predetermined site in the subject.

4. The measurement apparatus according to claim 1, wherein the processor is further configured to function as an observation time recording unit that records the first time period.

5. The measurement apparatus according to claim 1, wherein the processor is further configured to function as an image recording unit that records an observation image of the predetermined site at the first time.

6. The measurement apparatus according to claim 1, wherein
the time measurement unit comprises:
a first measurement unit that measures the first time period; and
a second measurement unit that measures a second time period during which the inside of the subject is subjected to a treatment,
wherein a difference between the first time period and the second time period is calculated as a second observation time period.

7. The measurement apparatus according to claim 6, wherein
the second measurement unit measures the second time period based on an operation signal in each of operation units provided in an endoscope system including an endoscope apparatus.

8. The measurement apparatus according to claim 6, wherein
the second measurement unit measures the second time period based on a notification of an operating state from a peripheral apparatus connected to an endoscope apparatus.

9. The measurement apparatus according to claim 1, wherein the first detection unit is configured to receive an input from a user and determines the first time based upon a timing of the receiving of the input from the user.

10. The measurement apparatus according to claim 1, wherein the second detection unit is configured to receive an input from a user and determines the second time based upon a timing of the receiving of the input from the user.

11. The measurement apparatus according to claim 1, wherein the time measurement unit is further configured to determine a second time period corresponding to a time not required for observation.

12. The measurement apparatus according to claim 11, wherein the time measurement unit is further configured to subtract the second time period from the first time period to determine an observation time period.

13. The measurement apparatus according to claim 12, wherein the non-observation time period is a period during which any one of a treatment apparatus, a water supply apparatus and a peripheral apparatus is operating.

14. The measurement apparatus according to claim 12, wherein the observation time period corresponds to a time period required for observation of the endoscopy procedure.

15. The measurement apparatus according to claim 1, wherein the predetermined site is a cecum of the subject.

16. A measurement method comprising:
measuring, after endoscopy is started, a first time period as a time period elapsed from when a distal end portion of an endoscope arrives at a predetermined site in a subject until the endoscopy ends;
measuring a second time period, the second time period being a time period within the first time period during which an inside of the subject is subjected to a treatment, and
calculating a difference between the first time period and the second time period as an observation time period.

17. A non-transitory, computer-readable recording medium having a program recorded thereon, the program causing a measurement unit that measures an observation time period of endoscopy to perform processing comprising:
a step of measuring a first time period as a time period elapsed from when a distal end portion of an endoscope arrives at a predetermined site in a subject until the endoscopy ends;
a step of measuring a second time period, the second time period being a time period within the first time period during which an inside of the subject is subjected to a treatment; and
a step of calculating a difference between the first time period and the second time period as the observation time period.
